(19) United States
(12) Patent Application Publication
(10) Pub. No.: US 2017/0319055 A1
(43) Pub. Date: Nov. 9, 2017

(54) ENDOSCOPIC ILLUMINATION SYSTEM

(71) Applicant: OLYMPUS CORPORATION, Tokyo (JP)

(72) Inventor: Takeharu INNAMI, Tokyo (JP)

(73) Assignee: OLYMPUS CORPORATION, Tokyo (JP)

(21) Appl. No.: 15/654,772

(22) Filed: Jul. 20, 2017

Related U.S. Application Data
(63) Continuation of application No. PCT/JP2015/000303, filed on Jan. 23, 2015.

(51) Int. Cl. A61B 1/07; G02B 23/24; A61B 1/00; A61B 1/06

(52) U.S. Cl. CPC A61B 1/07; G02B 23/2469; A61B 1/00172; A61B 1/0661; G02B 23/2446

(57) ABSTRACT

An endoscopic illumination system includes at least two light sources, an illumination system, a multiplexer, at least two detectors, and a light source controller. The multiplexer includes at least two input ports and an emission port. The multiplexer can propagate light that is input from the input ports to the emission port separately or after multiplexing the light. The multiplexer can output a portion of light propagating between the input ports and the emission port. The detectors detect the light propagating between the input ports and the emission port in each wavelength band. The light source controller adjusts the amount of light emitted from the light sources. The amount of light emitted from each light source is thereby appropriately adjusted even when multiple light sources are turned on simultaneously.

### CROSS-REFERENCE TO RELATED APPLICATION

The present application is a Continuing Application based on International Application PCT/JP2015/000303 filed on Jan. 23, 2015, the entire disclosure of which is incorporated herein by reference.

### TECHNICAL FIELD

This disclosure relates to an endoscopic illumination system that allows adjustment of the amount of illumination light.

### BACKGROUND

A known optical scanning endoscope apparatus is capable of capturing an image by oscillating an optical fiber that emits light to scan an object of observation. In order to adjust the amount of light irradiated onto the object of observation, it has been proposed to detect the amount of light leaking from the optical fiber (see JP 2011-255015 A (PTL 1)).

### CITATION LIST

### Patent Literature

PTL 1: JP 2011-55015 A

### SUMMARY

An endoscopic illumination system according to this disclosure includes:

at least two light sources configured to emit light of different wavelength bands;

an illumination system configured to illuminate an object of observation using the light emitted from the at least two light sources;

a multiplexer including at least two input ports optically connected respectively to the at least two light sources and an emission port optically connected to the illumination system, the multiplexer being capable of propagating light that is input through the at least two input ports to the emission port separately or after multiplexing the light and being capable of outputting a portion of light propagating between the input ports and the emission port;

at least two detectors configured to detect an amount of light in each wavelength band of the portion of light output by the multiplexer; and

a light source controller configured to adjust an amount of light emitted from the at least two light sources in accordance with the amount of light detected by the at least two detectors.

### BRIEF DESCRIPTION OF THE DRAWINGS

In the accompanying drawings:

FIG. 1 is a functional block diagram schematically illustrating the internal configuration of a scanning endoscope apparatus that includes an endoscopic lighting system according to one embodiment;

FIG. 2 is a functional block diagram schematically illustrating the internal configuration of the light source unit in FIG. 1;

FIG. 3 is a functional block diagram schematically illustrating the internal configuration of the illumination light detection unit in FIG. 2; and

FIG. 4 is an external view schematically illustrating the optical scanning endoscope body in FIG. 1.

### DETAILED DESCRIPTION

Embodiments are described below with reference to the drawings.

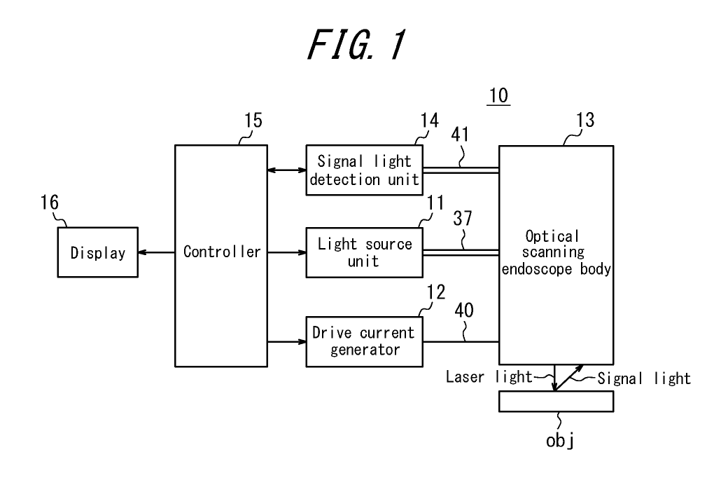

FIG. 1 is a functional block diagram schematically illustrating the internal configuration of a scanning endoscope apparatus that includes an endoscopic lighting system according to one embodiment. In the drawings, signal lines for transmitting signals and commands are indicated with solid lines, and light rays are indicated by dashed double-dotted lines.

A scanning endoscope apparatus 10 includes a light source unit 11, a drive current generator 12, an optical scanning endoscope body 13, a signal light detection unit 14, a controller 15, and a display 16. The endoscopic illumination system according to this embodiment includes the light source unit 11 and the below-described illumination system.

As described below, the light source unit 11 emits laser light and supplies the laser light to the optical scanning endoscope body 13. The drive current generator 12 transmits a drive signal necessary for scanning an object of observation obj to the optical scanning endoscope body 13. The optical scanning endoscope body 13 scans the object of observation obj with the laser light and propagates the signal light obtained by the scan to the signal light detection unit 14. The signal light detection unit 14 converts the propagated signal light to an electrical signal. The controller 15 synchronously controls the light source unit 11, the drive current generator 12, and the signal light detection unit 14, processes the electrical signal output by the signal light detection unit 14, synthesizes an image, and displays the image on the display 16.

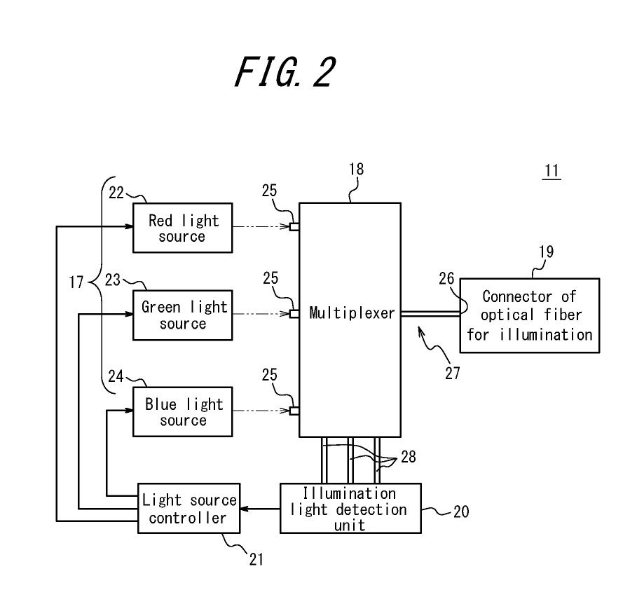

As illustrated in FIG. 2, the light source unit 11 is configured to include at least two light sources 17, a multiplexer 18, a connector 19 of an optical fiber for illumination, an illumination light detection unit 20, and a light source controller 21.

The at least two light sources 17 for example emit pulsed laser light of different wavelength bands. In this embodiment, the at least two light sources 17 are constituted by three light sources: a red light source 22, a green light source 23, and a blue light source 24. The red light source 22 may, for example, be a red laser that emits red laser light with a wavelength of 640 nm. The green light source 23 may, for example, be a green laser that emits green laser light with a wavelength of 532 nm. The blue light source 24 may, for example, be a blue laser that emits blue laser light with a wavelength of 445 nm. The at least two light sources 17 may emit a continuous wave (CW).

The multiplexer 18 includes at least two input ports 25 and an emission port 26. The at least two input ports 25 are optically connected respectively to the at least two light sources 17. In this embodiment, the at least two light sources 17 are constituted by three light sources. Therefore, the multiplexer 18 includes three input ports 25 respectively connected optically to the red light source 22, green light source 23, and blue light source 24. The input ports 25, red light source 22, green light source 23, and blue light source 24 are optically connected for example by space propagation and optical fiber propagation. The emission port 26 is optically connected to the below-described illumination system by the connector 19 of the optical fiber for illumination. The multiplexer 18 can propagate light that is input from the input ports 25 to the emission port 26 separately or after multiplexing the light. In the multiplexer 18, a portion of light propagating between the input ports 25 and the emission port 26 can be output to detectors in the illumination light detection unit 20.

The multiplexer 18 includes a light guide 27 for illumination and at least two light guides 28 for detection. The light guide 27 for illumination includes the emission port 26, and as described above, is connected to the illumination system by the connector 19 of the optical fiber for illumination. The light guides 28 for detection guide a portion of light propagating between the input ports 25 and the emission port 26 to the illumination light detection unit 20. The multiplexer 18 partitions the guided light at a predetermined ratio between the light guide 27 for illumination and the at least two light guides 28 for detection. The multiplexer 18 is, for example, an RGB combiner. The light guide 27 for illumination and the light guides 28 for detection may be configured by glass fiber. The light guides 28 for detection can guide a portion of the multiplexed light. The multiplexer 18 may be configured by combining various mirrors. The multiplexer 18 may also be configured to guide red laser light, green laser light, and blue laser light before multiplexing to the illumination light detection unit 20.

The connector 19 of the optical fiber for illumination optically connects to an illumination system provided in the optical scanning endoscope body 13 and supplies the laser light output from the multiplexer 18 to the optical fiber for illumination.

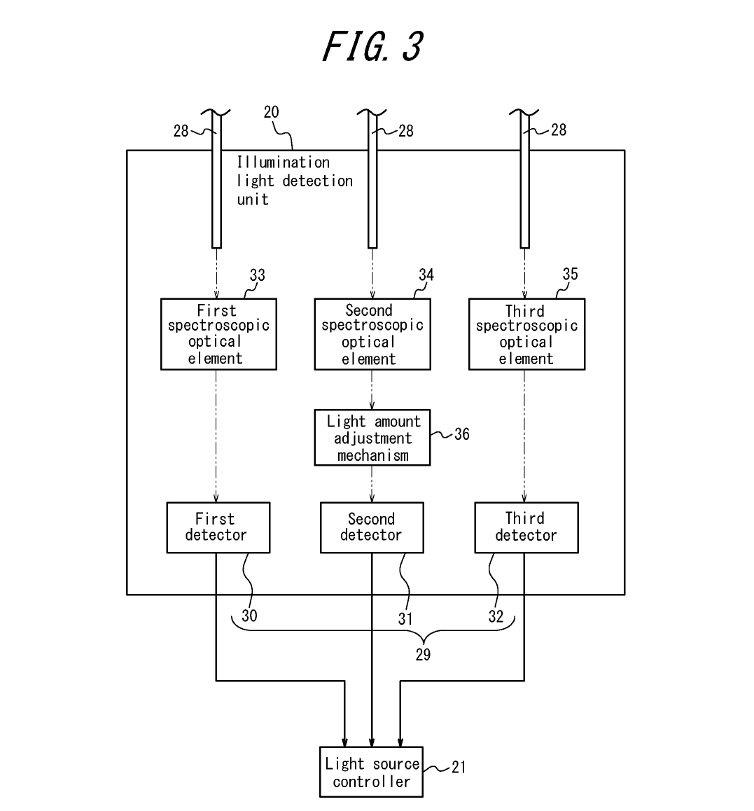

As illustrated in FIG. 3, the illumination light detection unit 20 has at least two detectors 29 that detect light of each wavelength band emitted from the at least two light sources 17. The at least two detectors 29 may, for example, be photodiodes. In this embodiment, the at least two light sources 17 are constituted by three light sources. Therefore, as the at least two detectors 29, a first detector 30, a second detector 31, and a third detector 32 are provided in the illumination light detection unit 20 to detect the amount of each of the red laser light, green laser light, and blue laser light emitted by the red light source 22, green light source 23, and blue light source 24.

The first detector 30 is optically connected to one of the light guides 28 for detection. A first spectroscopic optical element 33 is provided between the first detector 30 and the light guide 28 for detection. The first spectroscopic optical element 33 is a bandpass filter that, for example, transmits light in the red light band. Accordingly, the first detector 30 detects the amount of red light.

The second detector 31 is optically connected to another light guide 28 for detection. A second spectroscopic optical element 34 is provided between the second detector 31 and the light guide 28 for detection. The second spectroscopic optical element 34 is a bandpass filter that, for example, transmits light in the green light band. Accordingly, the second detector 31 detects the amount of green light.

The third detector 32 is optically connected to another light guide 28 for detection. A third spectroscopic optical element 35 is provided between the third detector 32 and the light guide 28 for detection. The third spectroscopic optical element 35 is a bandpass filter that, for example, transmits light in the blue light band. Accordingly, the third detector 32 detects the amount of blue light.

As necessary, the illumination light detection unit 20 may further include a light amount adjustment mechanism 36. The light amount adjustment mechanism 36 is disposed between one of the light guides 28 for detection and the corresponding detector. For example in this embodiment, the light amount adjustment mechanism 36 is disposed between the second spectroscopic optical element 34 and the second detector 31, reduces the amount of green light transmitted by the second spectroscopic optical element 34, and inputs the reduced green light into the second detector 31. The light amount adjustment mechanism 36 may, for example, be configured by a neutral density filter, by blocking a portion of the optical path to the detector, by adjusting the coupling efficiency between the light guide 28 for detection and the detector, or by providing the spectroscopic optical element with a specific transmittance.

The light source controller 21 (see FIG. 2) controls the at least two light sources 17, i.e. the red light source 22, green light source 23, and blue light source 24 in this embodiment, to adjust factors such as the amount of emitted light and the emission time. The light source controller 21 acquires the amount of each of the red laser light, green laser light, and blue laser light detected respectively by the first detector 30, the second detector 31, and the third detector 32. The light source controller 21 controls the at least two light sources 17 in accordance with each acquired amount of light. For example, when the ranges of the amount of red laser light, green laser light, and blue laser light are predetermined on the basis of measurement results and regulations, and one of the amounts of light to be detected falls below the lower limit of the range, then the light source controller 21 controls the light source to increase that amount of light. Also, when one of the amounts of light to be detected exceeds the upper limit of the range, the light source controller 21 controls the light source to reduce that amount of light and ultimately to turn that light source off.

In accordance with control by the controller 15, the drive current generator 12 (see FIG. 1) generates a drive signal that displaces the emission end of an optical fiber 37 for illumination in a spiral. The optical fiber 37 for illumination constitutes the illumination system. The drive current generator 12 supplies the drive signal to a driver provided in the optical scanning endoscope body 13.

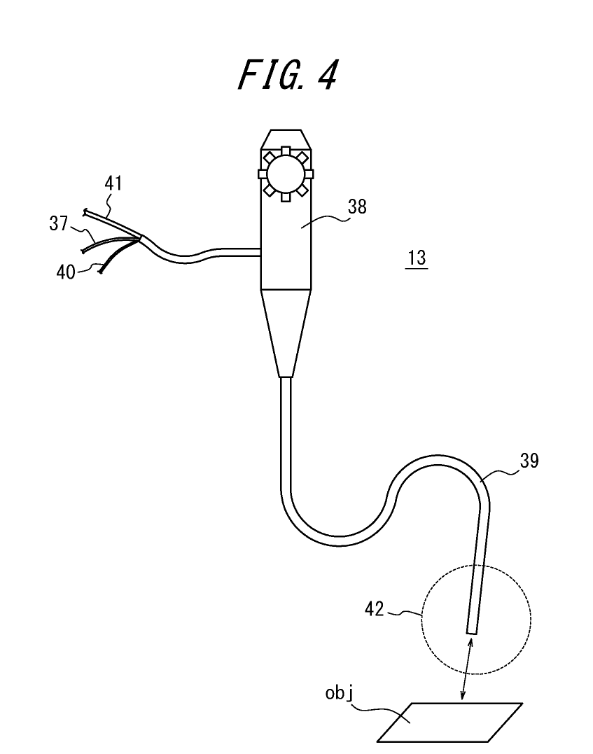

As illustrated in FIG. 4, the optical scanning endoscope body 13 includes an operation part 38 and an insertion part 39. One end of the operation part 38 is integrally connected to the base end of the insertion part 39.

The optical scanning endoscope body 13 includes the optical fiber 37 for illumination, a wiring cable 40, and an optical fiber bundle 41 for detection. The optical fiber 37 for illumination, wiring cable 40, and optical fiber bundle 41 for detection pass from the operation part 38 through the insertion part 39 and are drawn to a tip 42 (the portion enclosed by dashes in FIG. 4) of the insertion part 39.

The optical fiber 37 for illumination is connected to the connector 19 of the optical fiber for illumination in the light source unit 11 at the operation part 38 side and propagates laser light to the tip 42. The optical fiber 37 for illumination may, for example, be a single-mode fiber. A driver provided near the tip 42 vibrates the emission end of the optical fiber 37 for illumination in a spiral, thereby scanning an object for observation obj. A lens may be further provided at the tip 42, and the illumination system may be constituted by the optical fiber 37 for illumination and the lens.

The wiring cable 40 is connected to the drive current generator 12 at the operation part 38 side and transmits drive signals to the driver provided at the tip 42. The optical fiber bundle 41 for detection is connected to the signal light detection unit 14 at the operation part 38 side and propagates the signal light obtained at the tip 42 to the signal light detection unit 14.

The signal light detection unit 14 (see FIG. 1) includes a spectroscopic optical system and a red light detector, green light detector, and blue light detector. The spectroscopic optical system is configured by combining mirrors or filters and demultiplexes signal light into a red light component, a green light component, and a blue light component. The red light detector, green light detector, and the blue light detector are, for example, photomultiplier tubes or photodiodes and respectively detect the amount of light of the demultiplexed red light component, green light component, and blue light component.

The controller 15 controls each part of the scanning endoscope apparatus 10. For example, as described above, the controller 15 synchronously controls the light source unit 11, the drive current generator 12, and the signal light detection unit 14, processes the electrical signal output by the signal light detection unit 14, and synthesizes an image.

According to the endoscopic illumination system of this embodiment with the above configuration, the amount of light emitted from each of the at least two light sources 17 and multiplexed by the multiplexer 18 is detected in the respective wavelength band of the light source, and in accordance with this amount of light, the amount of light emitted from the light source is adjusted. Therefore, even if the amount of light emitted from one of the at least two light sources 17 rises abnormally to become a continuous wave, the light source in an abnormal state can be identified, and the amount of laser light illuminating the object of observation obj can be maintained in an appropriate range. In particular, as compared to a configuration that detects the amount of light using a single detector, this embodiment can reduce the effect of wavelength dependence on the power of each light source, the coupling efficiency and separation efficiency of the multiplexer 18, the transmission loss of the optical fiber forming part of the multiplexer 18, the transmittance of the first to third spectroscopic optical elements 33, 34, and 35, the transmittance of the light amount adjustment mechanism 36, and the light sensitivity of the first to third detectors 30, 31, and 32.

Since the endoscopic illumination system of this embodiment includes the light guide 27 for illumination and at least two light guides 28 for detection into which light is partitioned at predetermined ratios, the amount of light guided into the light guide 27 for illumination can be detected accurately using the light detected by the illumination light detection unit 20. Accordingly, the amount of laser light emitted from the optical fiber 37 for illumination can be maintained in an appropriate range.

The endoscopic illumination system of this embodiment also includes the light amount adjustment mechanism 36. The amount of light received by the detectors is therefore adjusted to be within a range detectable by the detectors. Accordingly, the amount of light in each wavelength band can be detected appropriately.

The endoscopic illumination system of this embodiment is applied to the optical scanning endoscope body 13 that performs scans with a simple structure using a single-mode fiber. Therefore, optical scanning by vibration of the fiber can always be performed with an accurate amount of light.

Although this disclosure has been described on the basis of embodiments and the accompanying drawings, it is to be noted that various changes and modifications will be apparent to those skilled in the art on the basis of this disclosure. Therefore, such changes and modifications are to be understood as included within the scope of this disclosure.

1. An endoscopic illumination system comprising:
at least two light sources configured to emit light of different wavelength bands;
an illumination system configured to illuminate an object of observation using the light emitted from the at least two light sources;
a multiplexer including at least two input ports optically connected respectively to the at least two light sources and an emission port optically connected to the illumination system, the multiplexer being capable of propagating light that is input through the at least two input ports to the emission port separately or after multiplexing the light and being capable of outputting a portion of light propagating between the input ports and the emission port;
at least two detectors configured to detect an amount of light in each wavelength band of the portion of light that propagates between the input ports and the emission port and is output by the multiplexer; and
a light source controller configured to adjust an amount of light emitted from the at least two light sources in accordance with the amount of light detected by the at least two detectors.

2. The endoscopic illumination system of claim 1, wherein the multiplexer comprises a light guide for illumination including the emission port and comprises at least two light guides for detection that guide the portion of light propagating between the input ports and the emission port to the at least two detectors.

3. The endoscopic illumination system of claim 2, further comprising a light amount adjustment mechanism configured to reduce light that is input to at least one of the at least two detectors.

4. The endoscopic illumination system of claim 1, wherein the illumination system includes a single-mode fiber, and the object of observation is scanned with light emitted from the single-mode fiber while the single-mode fiber is vibrated.
